(19) United States
(12) Patent Application Publication
(10) Pub. No.: US 2017/0127976 A1
(43) Pub. Date: May 11, 2017

(54) METHOD AND APPARATUS FOR IDENTIFICATION OF BIOMARKERS IN BREATH AND METHODS OF USNG SAME FOR PREDICTION OF LUNG CANCER

(71) Applicant: Michael PHILLIPS, Fort Lee, NJ (US)

(72) Inventor: Michael PHILLIPS, Fort Lee, NJ (US)

(21) Appl. No.: 15/263,621

(22) Filed: Sep. 13, 2016

Related U.S. Application Data
(63) Continuation-in-part of application No. 15/177,695, filed on Jun. 9, 2016.
(60) Provisional application No. 62/174,256, filed on Jun. 11, 2015.

(51) Int. Cl. A61B 5/08; A61B 5/00; G01N 30/72; A61B 5/097; G01N 33/497; H01J 49/00; A61B 10/00

(52) U.S. Cl. CPC A61B 5/082; H01J 49/0031; H01J 49/004; A61B 5/7275; A61B 10/0041; A61B 5/742; A61B 5/097; G01N 33/497; G01N 30/7206; A61B 2010/0087

(57) ABSTRACT

The present invention provides a method for identifying biomarkers and generating an output indicative of disease. The method for identifying biomarkers comprises the steps of collecting a breath sample from subjects known to have a disease and subjects known to be free of the disease; analyzing the collected breath samples to determine all mass ions in each of the collected breath samples using at least one time-resolved separation technique and at least one mass-resolved separation technique; identifying a subset of the determined mass ions in a processor as the biomarkers for detecting the disease, the subset of the determined mass ions are statistically significant for detecting the disease; and combining the subset of the determined mass ions in a multivariate algorithm in the processor to generate a value of a discriminant function indicating the likelihood that the subject has the disease.

### BACKGROUND OF THE INVENTION

The modern era of breath testing dawned in 1971, when Linus Pauling first reported that normal human breath contains large numbers of volatile organic compounds (VOCs) in low concentrations. Subsequent researchers have attempted to employ breath VOCs as disease biomarkers with varying degrees of success. The U.S. Food & Drug Administration (FDA) has approved a small number of breath tests for clinical use (e.g. breath nitric oxide for airways inflammation), but FDA has not yet approved a breath test for lung cancer. Despite 30 years of research resulting in more than 300 relevant publications, no breath VOC has yet emerged as a clinically useful biomarker of lung cancer when employed alone. However, several breath VOCs appear to provide moderately accurate biomarkers that could potentially identify lung cancer if combined with one another in a multifactorial algorithm.

In seeking breath biomarkers of lung cancer, researchers have employed a wide range of different tools including VOC separation methods using gas chromatography mass spectrometry (GC MS), non-separative detectors, such as electronic noses and chemosensors, analysis of expired breath condensate, measurement of breath temperature, and sniffer dogs. Analysis of breath VOCs with analytical instruments employing 2-dimensional GC has revealed a complex matrix of ˜2,000 different VOCs in a single sample. Data management tools for metabolomic analysis that were originally developed for genomics and proteomics have been used to manage the information. An increased risk of false discovery of biomarkers can arise when a multivariate model over-fits large number of candidate breath VOCs to a small number of test subjects, a pitfall that has been termed “voodoo correlations”, or “seeing faces in the clouds”.

Despite these concerns, breath biomarkers of lung cancer have been proposed as safe and cost-effective tools to help determine a person's risk of lung cancer. There is a clinical need for such a test because more people in the United States die from lung cancer than from any other type of cancer. Early detection can save lives: the National Lung Screening Trial found that screening with low-dose chest CT reduced mortality from lung cancer by 20%. However, the comparatively low positive predictive value (PPV) of chest CT (2.4% to 5.2%) has raised concerns that screening for lung cancer might yield an overwhelming number of false-positive test results.

Volatile organic compounds (VOCs) contained in human breath have been identified as candidate biomarkers of breast cancer as described in Phillips et al., Detection of an Extended Human Volatome with Comprehensive Two-Dimensional Gas Chromatography, Time-of-Flight Mass Spectrometry. PLoS One 2013; 8:e75274. The tool most widely employed for breath VOC biomarker discovery is gas chromatography mass spectrometry (GC MS). A sample of concentrated breath VOCs is injected onto a chromatographic column that separates the complex mixture into a series of individual VOCs according to their physicochemical properties such as polarity and boiling point. The separated VOCs then flow into a detector where they are broken into fragments by a beam of high-energy electrons in a vacuum, and the resulting mass spectrum of fragments comprises a “fingerprint” that can be used to identify the VOC from a computer-based spectral library.

GC MS is a widely accepted tool, but it can potentially yield erroneous identification of analytes if a mixture as complex and diverse as human breath VOCs overburdens the separation column. If the separation of VOCs is incomplete, then two or more VOCs may enter the MS detector simultaneously, and their combined mass spectra may lead to misidentification of their chemical structures in the spectral library. Breath volatile organic compounds (VOCs) contain biomarkers of breast cancer that are detectable with gas chromatography mass spectrometry (GC MS). However, chemical identification of breath VOC biomarkers may be erroneous because spectral matching can misidentify their structure.

It is desirable to provide new and improved methods of identifying biomarkers of a disease to potentially improve the sensitivity and specificity of the disease screening and reduce the number of false-positive and false-negative test findings.

### SUMMARY OF THE INVENTION

The present invention provides a method for identifying biomarkers and generating an output indicative of disease, including for example lung cancer or breast cancer. The method for identifying biomarkers comprises the steps of:

collecting a breath sample from subjects known to have a disease and subjects known to be free of the disease;

analyzing the collected breath samples to determine all mass ions in each of the collected breath samples using at least one time-resolved separation technique and at least one mass-resolved separation technique;

identifying a subset of the determined mass ions in a processor as the biomarkers for detecting the disease, the subset of the determined mass ions are statistically significant for detecting the disease; and

combining the subset of the determined mass ions in a multivariate algorithm in the processor to generate a value of a discriminant function indicating the likelihood that the subject has the disease. It will be appreciated that the subset of the determined mass ions will be different for each disease which is analyzed with the collected breath sample. Similarly, each subset of determined mass ions for each disease is combined in a different multivariate algorithm in order to generate a value of the discriminant function for the particular disease.

In one embodiment, biomarker mass ions are determined from breath VOCs after bombardment of the breath VOCs with high energy electrons using a mass spectrometer.

The invention also comprises a method for predicting the probable presence of disease in a test subject using the method for identifying biomarkers described above. In one embodiment, the method of the present invention is used for predicting the probable presence of lung cancer in a test subject using the method for identifying biomarkers of the present invention. In another embodiment, the method of the present invention is used for predicting the probable presence of breast cancer in a test subject using the method for identifying biomarkers of the present invention.

Another embodiment of the invention features a system for identifying a plurality of biomarkers for predicting a disease in a subject including an apparatus for collecting a breath sample from subjects known to have the disease and subjects known to be free of the disease. A mass spectrometer (MS) associated with a gas chromatograph (GC) apparatus analyzes the collected breath samples to determine all mass ions in each of the collected breath samples. A computer identifies a subset of the determined mass ions as the biomarkers for detecting the disease, the subset of the determined mass ions are statistically significant for detecting the disease, and combines the subset of the determined mass ions in a multivariate algorithm to generate a discriminant function. The discriminant function indicates a value of the likelihood that the subject has the disease. In particular embodiments, the system can be used for predicting the probable presence of lung cancer or breast cancer in the subject using the identified biomarkers for predicting respectively lung cancer or breast cancer in the multivariate algorithm.

It was found that biomarkers determined with the method of the present invention accurately predicted lung cancer in a blinded replicated study. Breath testing in parallel with chest CT can potentially improve the accuracy of lung cancer screening.

It was found that biomarkers determined with the method of the present invention accurately identified women with breast cancer and can be used for early diagnosis and treatment monitoring.

### BRIEF DESCRIPTION OF THE DRAWINGS

The invention will be more fully described by reference to the following drawings.

FIG. 1 is flow diagram of a method for identifying biomarkers and generating an output indicative of predicting disease in accordance with the teachings of the present invention.

FIG. 2 is a flow diagram of steps which can be used for identifying a subset of mass ions which are statistically significant for detecting disease.

FIG. 3 is a flow diagram of steps which can be used for selecting the biomarker mass ions with at least greater than random diagnostic accuracy.

FIG. 4A is a graph of the number of single ions detecting lung cancer versus the area under curve (AUC) of its associated receiver operating characteristic (ROC) curve for all single ions detecting lung cancer. This displays the outcome of correct assignment of diagnosis as well as a Monte Carlo simulation of the outcome of random assignment of diagnosis.

FIG. 4B is a graph of the number of single ions detecting breast cancer versus the area under curve (AUC) of its associated receiver operating characteristic (ROC) curve for all single ions detecting breast cancer. This displays the outcome of correct assignment of diagnosis as well as a Monte Carlo simulation of the outcome of random assignment of diagnosis.

FIG. 5 is a schematic diagram a system for identifying a plurality of biomarkers for detecting disease and for detecting disease in accordance with the teachings of the present invention.

FIG. 6A is a total ion chromatogram generated by mass spectrometry of a breath sample.

FIG. 6B is a mass spectrum of ions in a chromatograph peak of the chromatogram.

FIG. 7 is a flow diagram of an experimental protocol including an un-blinded phase and a blinded phase for identifying biomarkers and generating an output indicative of detecting lung cancer.

FIG. 8A is a plot of the values of a selected subset of single ion biomarkers versus retention time on the chromatogram.

FIG. 8B is a receiver operating characteristic (ROC) curve for detecting lung cancer in the unblinded phase.

FIG. 9A is a plot of discriminant function DF values at laboratory A versus discriminant function DF values at laboratory B in the blinded phase.

FIG. 9B is a plot of predicted sensitivity and specificity in subjects with biopsy-proven lung cancer and chest CT negative for lung cancer.

FIG. 9C is a graph of the value of the discriminant function (DF) versus the percentage of subjects detected with lung cancer

FIG. 9D a graph of receiver operating characteristic (ROC) curves for detecting lung cancer in the blinded-phase ROC curves of the predicted outcomes of the method of the present invention.

FIG. 10A is a graph of expected outcome of chest CT combined with breath testing performed in accordance with the method of the present invention.

FIG. 10B is a graph of a positive predictive value (PPV) of chest CT combined with breath testing performed in accordance with the method of the present invention.

FIG. 10C is a graph of expected outcome of chest CT combined with breath testing performed in accordance with the method of the present invention.

FIG. 11A is a plot of predicted sensitivity and specificity in a training set of subjects for predicting breast cancer.

FIG. 11B is a plot of predicted sensitivity and specificity in a test set of subjects for predicting breast cancer.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

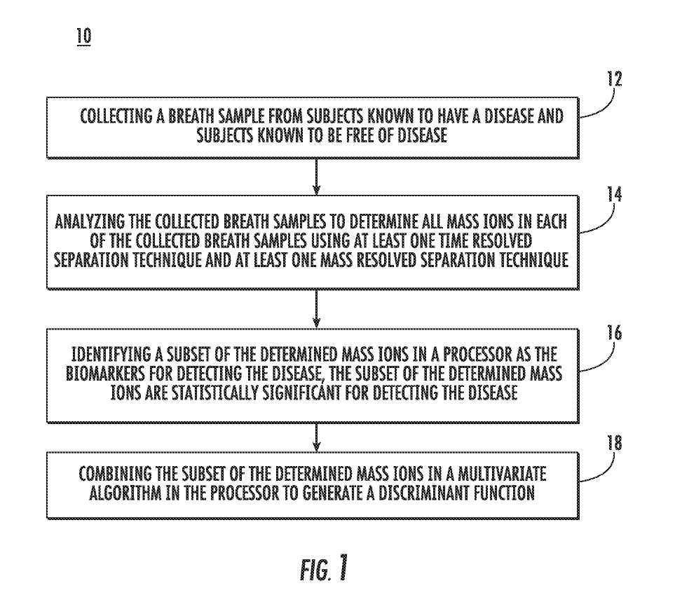

FIG. 1 is flow diagram of a method 10 for identifying biomarkers and generating an output indicative of a disease. In block 12, breath samples are collected from subjects known to have the disease and subjects known to be free of the disease. In one embodiment, breath samples are collected from subjects known to have lung cancer. In an alternative embodiment, breath samples are collected from subjects known to have breast cancer.

In block 14, the collected breath samples are analyzed to determine all mass ions in each of the collected breath samples using at least one time-resolved separation technique and at least one mass-resolved separation technique. In a preferred embodiment, the samples are analyzed with gas chromatography and mass spectrometry (GC MS). Data from the GC MS of chromatograms is processed in a computer processor to identify mass ions in the sample.

In block 16, a subset of the determined mass ions which are statistically significant for detecting disease are identified as the biomarkers for detecting the disease. In block 18, the subset of the determined mass ions is combined in a multivariate predictive algorithm to generate a value of a discriminant function (DF) indicating the likelihood that the subject has a disease. For example, a subset of determined mass ions can be determined which are statistically significant for detecting lung cancer or breast cancer.

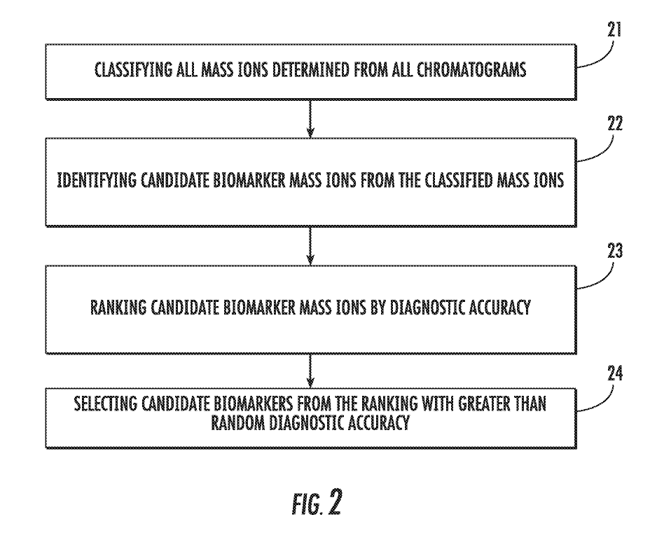

FIG. 2 is a flow diagram of steps which can be used for identifying the subset of mass ions which are statistically significant for detecting a disease. In block 21, all mass ions determined from all chromatograms of the breath samples are classified using intensities and retention times. Candidate biomarker mass ions from the classified mass ions are identified in block 22. The candidate biomarker mass ions are ranked by diagnostic accuracy for predicting the disease in block 23. In block 24, the candidate biomarker mass ions with at least greater than random diagnostic accuracy are selected as the subset of mass ions which are statistically significant for detecting the disease.

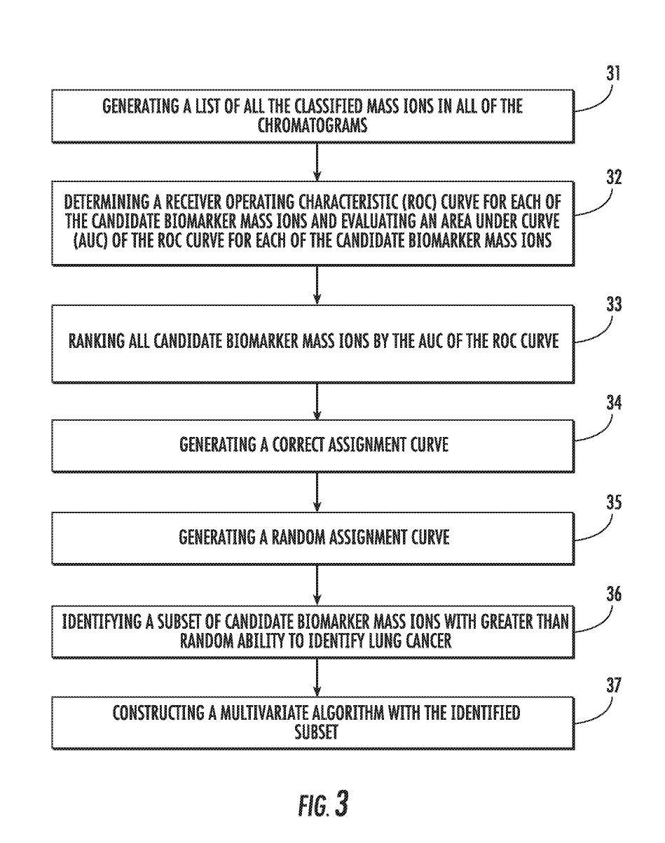

FIG. 3 is a flow diagram of steps which can be used for selecting the biomarker mass ions with at least greater than random diagnostic accuracy to be used in the multivariate predictive algorithm. In block 31, a list is generated of all the classified mass ions in all of the chromatograms. In block 32, the diagnostic accuracy is determined by determining a receiver operating characteristic (ROC) curve for each of the candidate biomarker mass ions and evaluating an area under curve (AUC) of the ROC curve for each of the candidate biomarker mass ions reflecting the sensitivity and specificity for predicting the disease. In block 33, all candidate biomarker mass ions are ranked by the AUC of the ROC curve. The ranking can be from highest to lowest.

Blocks 34, 35 and 36 describe steps using multiple Monte Carlo simulations to identify a set of mass ion biomarkers of a disease that detect the disease with greater than random accuracy. In block 34, a correct assignment curve is constructed with data of the AUC of the ROC curves for all candidate biomarker mass ions. In one embodiment, block 34 can be performed by assigning all data of the AUC of the ROC curves to a series of bins with incremental values. For example, the bins can be assigned values of 0.50 to 0.51, 0.51 to 0.52 and so forth up to 0.99 to 1.0. The correct assignment curve is generated as a plot of the number of mass ions in a bin on the y-axis versus the AUC value of a bin on the x-axis

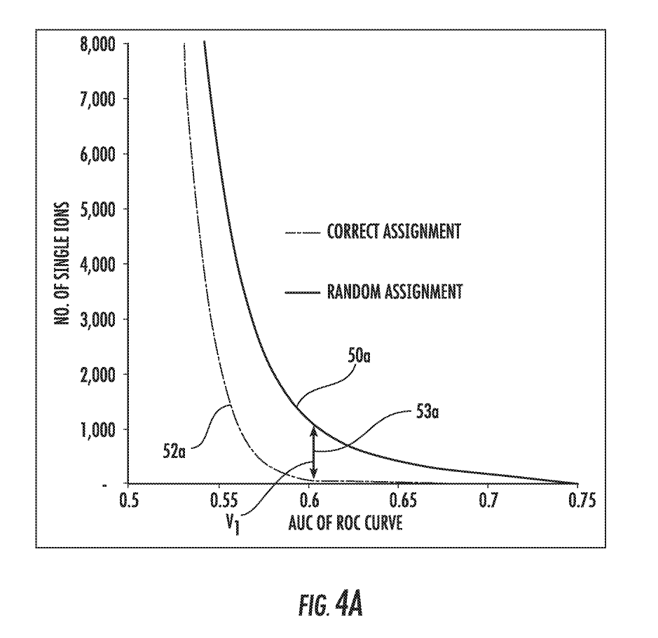

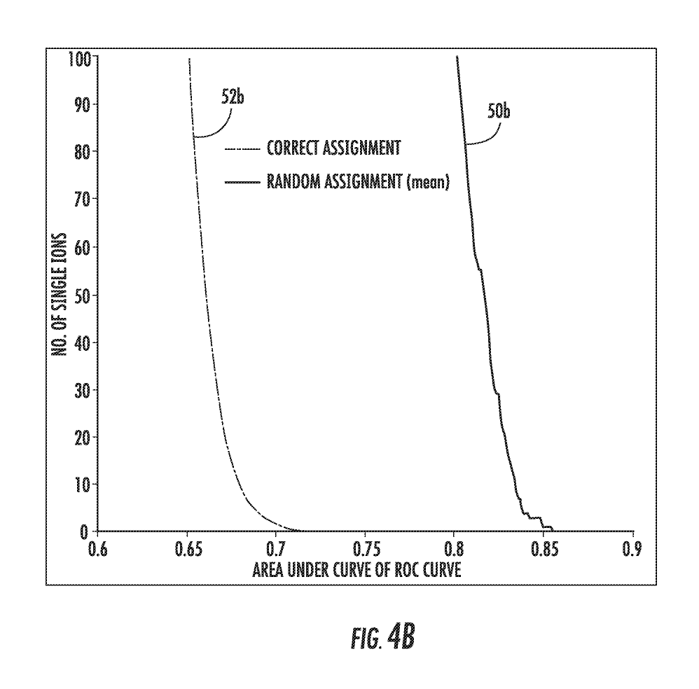

An example correct assignment curve for lung cancer is shown as 50a in FIG. 4A. A list of more than 70,000 candidate mass ion biomarkers of lung cancer was obtained from a series of 5 sec segments in aligned chromatograms.

The accuracy of the correct assignment curve can be re-evaluated by comparison of Monte Carlo simulations of the identified subset of mass ions to a plurality of Monte Carlo simulations of random assignment of each of the mass ions to either disease or being free of disease. Referring to FIG. 3, in block 35, Monte Carlo simulations are used to generate a random assignment curve. The random assignment curve is generated by randomly assigning each mass ion on the list of all classified mass ions to a group of either a mass ion of disease or a mass ion free of disease. The diagnostic accuracy of each of the randomly assigned mass ions is determined by the AUC of its ROC. The random assignment and determination of the diagnostic accuracy for the randomly assigned mass ion is repeated a predetermined number of times, for example the steps can be repeated at least 40 times. All data of the AUC of the ROC curves is assigned to a series of bins with incremental values. For example, the bins can be assigned values of 0.50 to 0.51, 0.51 to 0.52 and so forth up to 0.99 to 1.0. The random assignment curve is generated as a plot of the number of mass ions in a bin on the y-axis versus the AUC value of a bin on the x-axis. An example random assignment curve is shown as 52a for lung cancer in FIG. 4A.

Referring to FIG. 3, in block 36, the subset of candidate biomarker mass ions with greater than random ability to identify disease are identified using a correct assignment curve and a random assignment curve. In one embodiment, the subset of candidate biomarker mass ions with greater than random ability to identify lung cancer are identified using correct assignment curve 50a and the random assignment curve 52a as shown in FIG. 4A. In one embodiment, the subset of candidate biomarker mass ions with greater than random ability to identify breast cancer are identified using correct assignment curve 50b and the random assignment curve 52b as shown in FIG. 4B.

In one embodiment, block 36 can be implemented using vertical line V₁ 53a of FIG. 4A generated at the point where the value of the random assignment curve 52a is zero. The point at which vertical line V₁ 53a intersects correct assignment curve 50a identifies candidate biomarker mass ions with greater than random ability to identify disease. In this embodiment, the area under the ROC curve for each of the selected candidate biomarker mass ions having greater than random diagnostic accuracy is at least 0.6.

Referring to FIG. 3, In block 37, the multi-variate predictive algorithm is constructed using the candidate biomarker mass ions from the correct assignment curve that were identified as having greater than random ability to identify disease. A list is generated of all candidate biomarker mass ions in the correct assignment curve that were identified as having greater than random ability to identify the disease. Each of the listed candidate mass ions are ranked by the AUC of the ROC curve. The ranking can be from highest to lowest. A predetermined number of candidate biomarker mass ions having the highest ranking are used to generate the multivariate predictive algorithm. For example, the multivariate predictive algorithm can be employed to predict the diagnosis of lung cancer or breast cancer.

For example, from the embodiment shown in FIG. 4A for detecting lung cancer, the top 200 mass ions having the highest ranking are used to generate the multivariate predictive algorithm. For example, from the embodiment shown in FIG. 4B for detecting breast cancer, the top 200 mass ions having the highest ranking are used to generate the multivariate predictive algorithm

Method 10 for identifying biomarkers and generating an output indicative of disease of the present invention can be used to detect the probable presence of a disease in a human subject. A breath sample from a test subject is collected, chemically analyzed and the data is analyzed with the multivariate algorithm to generate a value of the discriminant function for the test subject. The value of the discriminant function for the test subject is compared to the value of the discriminant function determined in block 18.

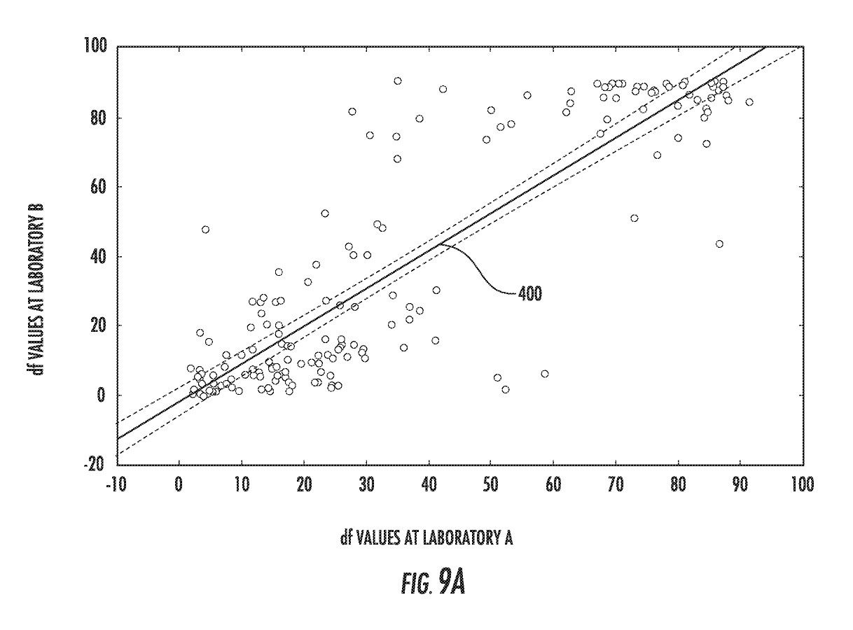

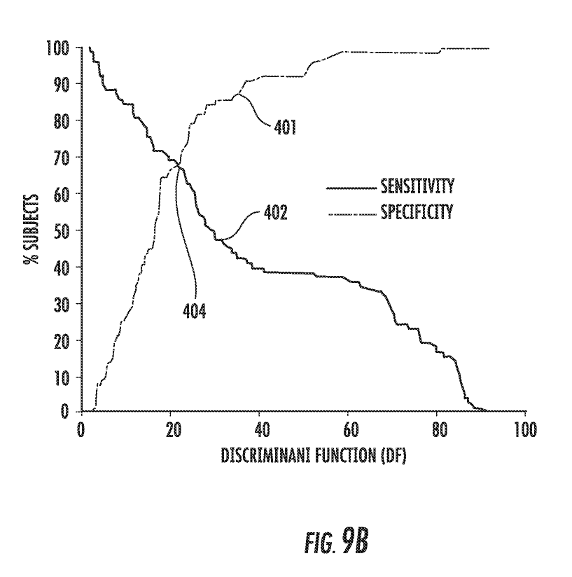

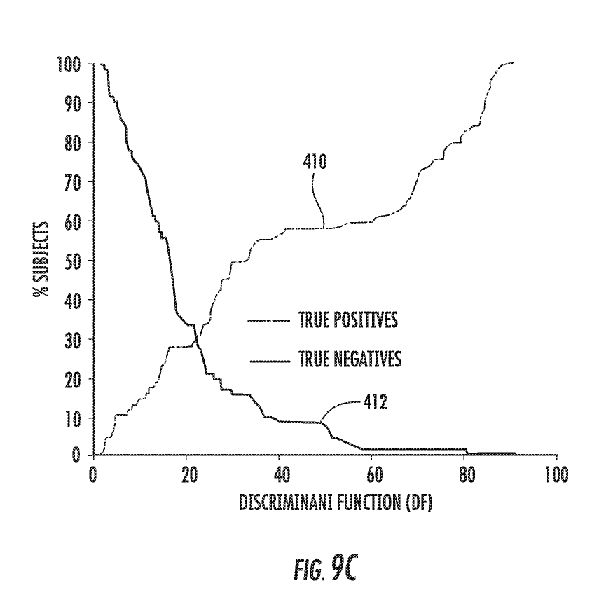

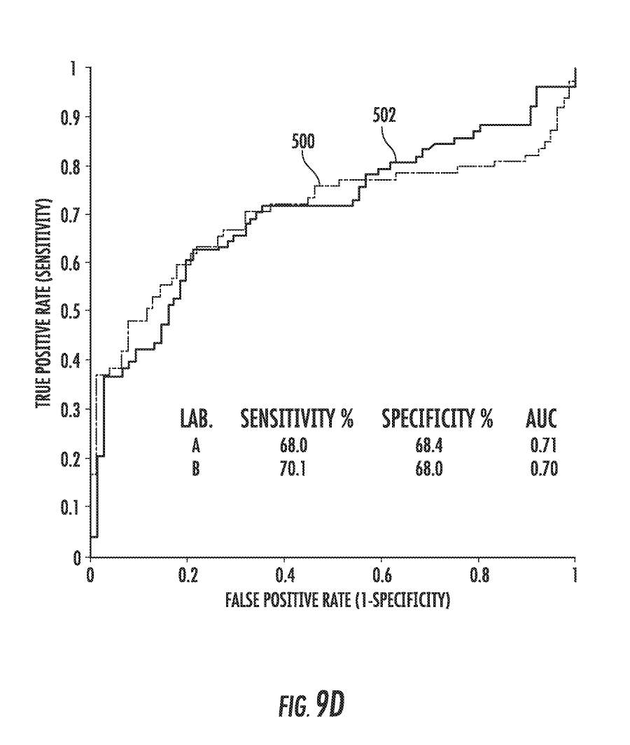

In one example, the probability of presence of lung cancer in a test subject increases with the value of the discriminant function, as shown in FIG. 9C. Method 10 for identifying biomarkers and generating an output indicative of lung cancer can be combined with results from screening of the test subject with a chest CT scan. When the two tests are combined, the resulting sensitivity and specificity is potentially greater than the sensitivity and specificity of either test employed alone.

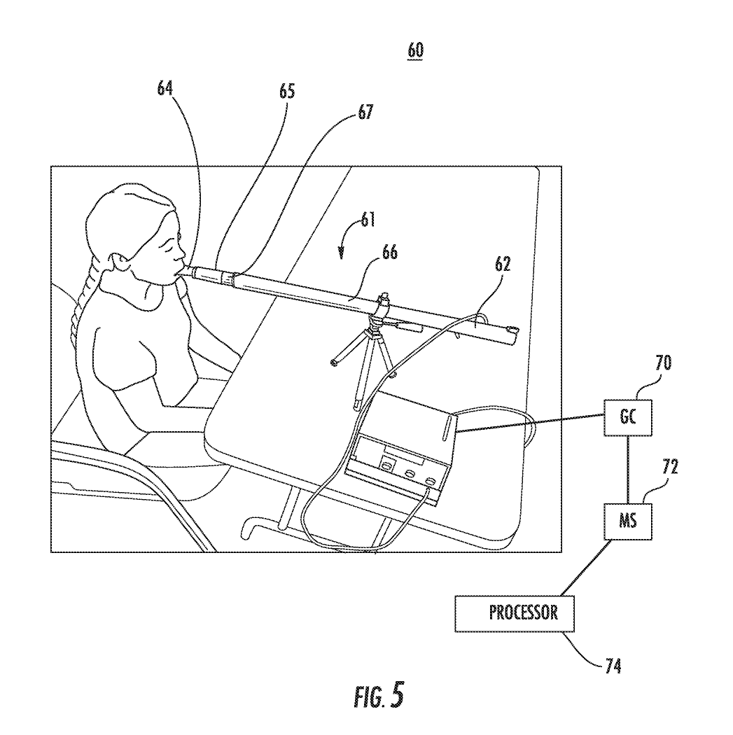

FIG. 5 is a schematic diagram a system for identifying a plurality of biomarkers for detecting disease and for detecting disease 60 in accordance with the teachings of the present invention. Breath collection apparatus (BCA) 61 collects samples of volatile organic compounds (VOCs) in alveolar breath and in air onto separate sorbent traps 62. The subject breathes through a disposable valved mouthpiece 64 and a bacterial filter 65. For example, the subject breathes normally for 2.0 min into breath collection apparatus (BCA) 61. Breath reservoir 66 separates alveolar from dead space breath, and alveolar breath is pumped from reservoir 66 through sorbent trap 62. A suitable breath reservoir 66 is a stainless steel tube packed with two grades of activated carbon to capture the VOCs in breath. For example, breath reservoir 66 can capture 1.0 l of breath. A 1.0 l sample of room air is also collected onto second trap 67. A new disposable valved mouthpiece 64 and bacterial filter 65 is employed for every breath collection. For example, each subject can donate two samples for replicate assay at two independent laboratories. An example breath collection apparatus (BCA) is described in U.S. Pat. No. 6,726,637, hereby incorporated by reference into this disclosure.

VOCs are thermally desorbed from the sorbent trap 62, separated by gas chromatography apparatus 70, and injected into mass spectrometry detector 72. In mass spectrometry detector 72 the VOCs are bombarded with energetic electrons in a vacuum and degraded into a set of ionic fragments, each with its own mass/charge (m/z) ratio. Data from gas chromatography apparatus 70 and mass spectrometry detector 72 is received at processor 74.

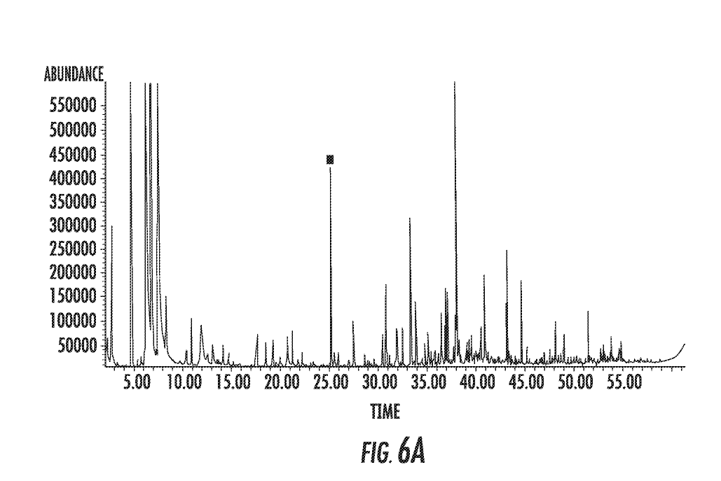

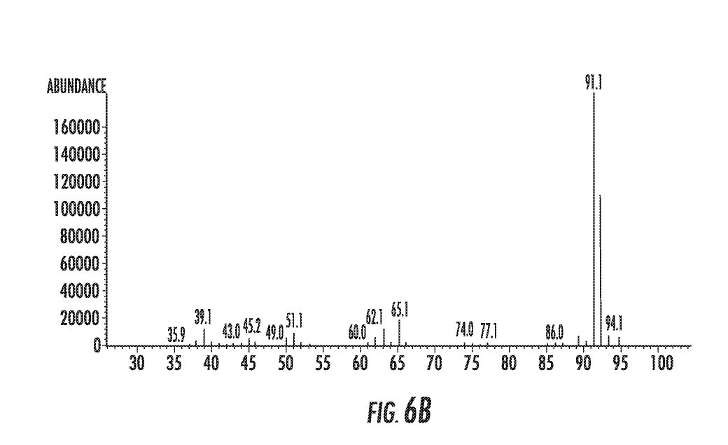

FIG. 6A is an example total ion chromatogram total ion current as a function of time, as a series of VOCs enter the detector sequentially. The total ion current from a peak containing toluene is marked, and the mass spectrum of the constituent single ions is shown in the lower panel. A typical total ion chromatogram derived from a sample of human breath VOCs usually displays ˜150 to 200 separate peaks is shown in FIG. 6A. A mass spectrum of ions in a chromatogram peak of the chromatogram is shown in FIG. 6B.

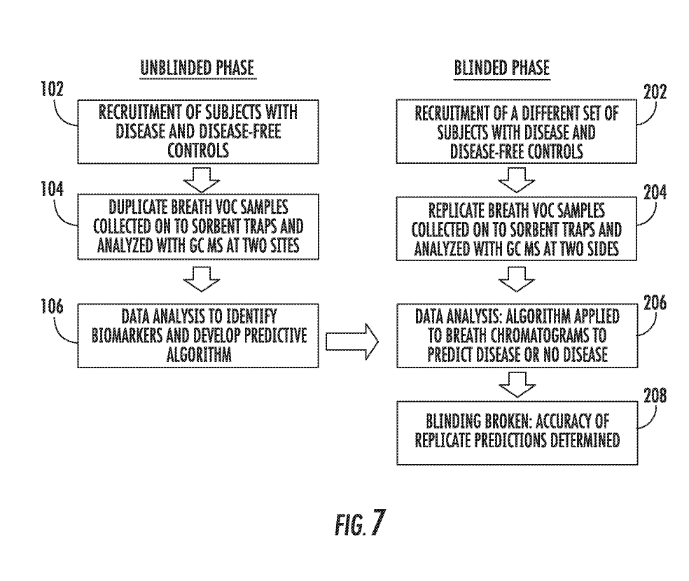

FIG. 7 is a flow diagram of an experimental protocol including an unblinded phase and a blinded phase to cross-validate the predictive algorithm. In the unblinded model-building phase, subjects were recruited in block 102. Breath samples from subjects with a disease, for example lung cancer or breast cancer, and from disease-free controls were analyzed with a highly sensitive and selective GC MS assay in block 104. A statistical method identified a set of non-random breath biomarkers of the disease that were then employed in a multivariate predictive algorithm to generate a value of a discriminant function (DF) indicating the likelihood that the subject has the disease, for example lung cancer or breast cancer, in block 106. In the blinded model-testing phase, a different set of subjects was recruited in block 202 to predict the disease, for example lung cancer or breast cancer, in a different set of subjects. All breath assays and disease predictions can be replicated in independent analytical laboratories in block 204. In block 206, data of breath chromatograms was analyzed to predict disease, for example cancer or no cancer, in a subject using the multivariate predictive algorithm to generate the value of a discriminant function (DF). In block 208, accuracy of replicate predictions can be determined.

Although some embodiments herein refer to methods, it will be appreciated by one skilled in the art that they may also be embodied as a system or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “processor,” “device,” or “system.” Furthermore, aspects of the present invention may take the form of a computer program product embodied in one or more computer readable mediums having computer readable program code embodied thereon. Any combination of one or more computer readable mediums may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code embodied on a computer readable medium may be transmitted using any appropriate medium, including but not limited to CDs, DVDs, wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks. The flowcharts and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

Methods

Model-Building Phase—Unblinded for Detecting Lung Cancer

In the unblinded model-building phase, breath VOCs were analyzed with gas chromatography mass spectrometry to provide data in breath chromatograms. The human subjects from which breath chromatograms were obtained are shown in Table 1 which included Group 1: 82 asymptomatic high-risk including smokers aged >=50 years of age undergoing chest CT; Group 2: 84 symptomatic high-risk subjects with a tissue diagnosis; Group 3: 99 symptomatic high-risk subjects without a tissue diagnosis; and Group 4: 35 apparently healthy subjects free of lung cancer.

Multiple Monte Carlo simulations identified candidate breath VOC mass ions from the data with greater than random diagnostic accuracy for detecting lung cancer, and the determined candidate biomarkers were combined in the multivariate predictive algorithm.

In the blinded model-testing phase, breath VOCs were analyzed in a new set of human subjects. The subjects from which breath chromatograms were obtained included Group 1: 68 asymptomatic high-risk including smokers aged >=50 years of age undergoing chest CT; Group 2: 51 symptomatic high-risk subjects with a tissue diagnosis; Group 3: 76 symptomatic high-risk subjects without a tissue diagnosis; and Group 4: 19 apparently healthy subjects free of lung cancer. The multivariate algorithm predicted discriminant function (DF) values in blinded replicate samples analyzed independently at two laboratories (A and B).

TABLE 1
Human Subjects

| | | | | |
|---|---|---|---|---|
| Group 1 | Group 2 | Group 3 | | |
| Asymptomatic | Symptomatic | Symptomatic | Group 4 | |
| high-risk smokers | high-risk | high-risk | Healthy | |
| Chest CT | No tissue diagnosis | With tissue diagnosis | normals | Total |

Model-building phase
Unblinded

| | | | | | |
|---|---|---|---|---|---|
| No. | 82 | 84 | 99 | 35 | 300 |
| Age: mean yr (SD) | 61.82 (7.24) | 64.58 (8.90) | 67.72 (10.74) | 44.46 (13.72) | |
| Tobacco smoking: | 42.10 (16.89) | 36.65 (24.45) | 48.49 (28.00) | 18.38 (15.83) | |
| mean pack years (SD) | | | | | |
| Male/female | 40/41 | 30/54 | 49/52 | 9/26 | |
| Lung cancer | | | | | |
| positive | 1 | | 94 | | |
| negative | 81 | | 4 | | |
| not reported | | | 1 | | |

Model-testing phase
Blinded

| | | | | | |
|---|---|---|---|---|---|
| No. | 68 | 51 | 76 | 19 | 214 |
| Age: mean yr (SD) | 62.15 (7.46) | 62.78 (12.08) | 66.48 (8.90) NS* | 49.11 (13.96) | |
| Tobacco smoking: | 43.81 (22.56) | 36.34 (31.40) | 51.59 (36.60) NS* | 11.66 (7.57) | |
| mean pack years (SD) | | | | | |
| Male/female | 30/38 | 26/25 | 32/43 | 9/10 | |
| Lung cancer | | | | | |
| positive | 3 | | 73 | | |
| negative | 65 | | 0 | | |
| not reported | | | 3 | | |

*NS compared to Group 1 (2-tailed t-test assuming equal variances)

The subjects of Group 3 are shown in Table 2.

TABLE 2

| | Model-building | Model-testing |
|---|---|---|
| | phase | phase |
| Group 3 tissue diagnosis | Unblinded | Blinded |
| Adenocarcinoma | 52 | 47 |
| Adenocarcinoma with | 3 | 0 |
| bronchioloalveolar carcinoma | | |
| Bronchioloalveolar carcinoma | 1 | 4 |
| Carcinoid | 2 | 0 |
| Small cell lung carcinoma | 1 | 0 |
| Squamous cell lung carcinoma | 16 | 13 |
| Other or unspecified | 1 | 1 |
| Other or unspecified | 16 | 8 |
| non-small cell lung carcinoma | | |
| Mesothelioma | 2 | 0 |
| Total | 94 | 73 |

Collection of breath VOC samples: Collection of breath VOC samples was performed in accordance with method 10 for identifying biomarkers and generating an output indicative of lung cancer and system 60. A subject wears a nose clip and breathes normally through a disposable valved mouthpiece and bacterial filter into the BCA for 2.0 min. Alveolar breath VOCs are captured on to a sorbent trap that is immediately sealed in a hermetic container. Since there is low resistance to expiration (˜6 cm water), breath samples could be collected without discomfort from elderly patients and those with respiratory disease. In order to minimize the risk of potential site-dependent confounding factors such as environmental contamination of room air, subjects in all four groups donated breath samples in the same room at each clinical site. All subjects donated two samples for replicate assay at two independent laboratories (Menssana Research, Inc and American Westech, Inc., Harrisburg, Pa.). Samples were stored at −15° C. prior to analysis.

Analysis of breath VOC samples: Analysis of breath VOC sample was performed with method 10 for identifying biomarkers and generating an output indicative of lung cancer and system 60. Using automated instrumentation, VOCs were thermally desorbed from the sorbent trap 62, cryogenically concentrated, and assayed by gas chromatography mass spectrometry (GC MS). A known quantity of an internal standard (bromofluorobenzene) was automatically loaded on to all samples in order to normalize the abundance of VOCs and to facilitate alignment of chromatograms. A typical total ion chromatogram of breath VOCs is shown in FIG. 4B. Single ions detected in a typical chromatograph peak are shown in FIG. 4C.

Analysis of data: GC MS data from both laboratories was pooled for analysis and development of a single predictive algorithm.

Alignment of single ion masses in chromatograms: Chromatograms were processed with metabolomic analysis software (XCMS in R) in order to generate a table listing retention times with their associated ion masses and intensities. Retention times and ion mass intensities were normalized to the bromofluorobenzene (ion mass 95) internal standard in each chromatogram. The aligned data was then binned into a series of 5 sec retention time segments.

Identification of biomarker single ions: The statistical methods have been previously described. Mass ions as candidate biomarkers of lung cancer were ranked by comparing their intensity values in subjects with lung cancer (Group 3 lung cancer confirmed by tissue diagnosis shown in table 3) to cancer-free controls (Group 1 with negative chest CT). In each 5 sec time segment, the diagnostic accuracy of each mass ion was ranked according to its C-statistic value [(area under curve (AUC) of the receiver operating characteristic (ROC) curve]. Multiple Monte Carlo simulations were employed in order to minimize the risk of including random identifiers of disease by selecting the mass ions in each time segment that identified active lung cancer with greater than random accuracy. The average random behavior of mass ions in each time segment was determined by randomly assigning subjects to the “lung cancer” or the “cancer-free” group and performing 40 estimates of the C-statistic. For any given value of the C-statistic, it was then possible to identify the ionic biomarkers that exhibited greater diagnostic accuracy with correct assignment than with multiple random assignments.

Development of predictive algorithm: Biomarker ions that identified lung cancer with greater than random accuracy were employed to construct a predictive algorithm using multivariate weighted digital analysis (WDA).

Model-Testing Phase—Blinded for Detecting Lung Cancer

Blinding procedures: The independent monitor maintained a database of all clinical and diagnostic data, and this information was not shared with any participant in the research. Laboratories received no clinical information and only the subject identification number accompanied sorbent traps sent for analysis.

Human subjects: A new set of human subjects was recruited in the same fashion as described above in the model-building phase. No subject from the unblinded phase was included in the blinded phase of the research.

Collection of breath VOC samples and analysis of breath VOC samples were performed in the same fashion as described above in the model-building phase.

Prediction of outcomes: The predictive algorithm developed in the unblinded phase was applied to the mass ions in each of the blinded breath chromatograms in order to generate a discriminant function (DF) value. This procedure was replicated in duplicate breath samples that were analyzed at two laboratories. At the conclusion of the study, the resulting DF values with their associated subject identification numbers were transmitted to the monitor who then broke the blinding and determined the predictive accuracy of the breath test. There were no adverse effects associated with breath testing in either phase of the study.

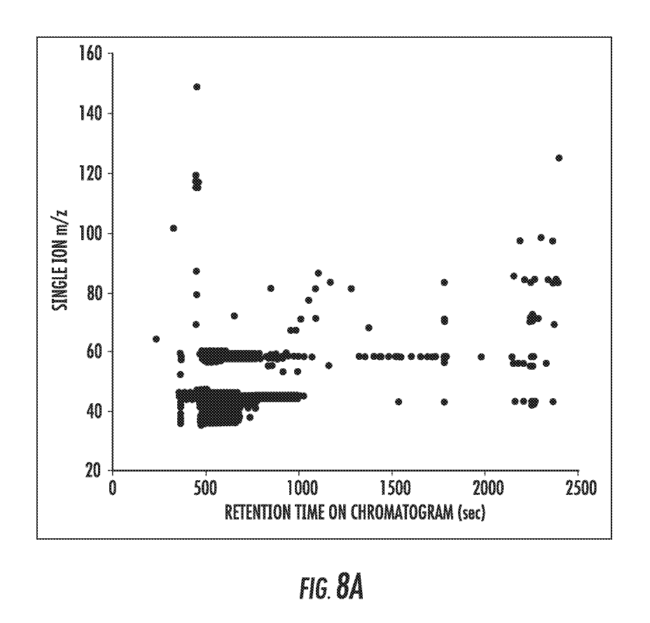

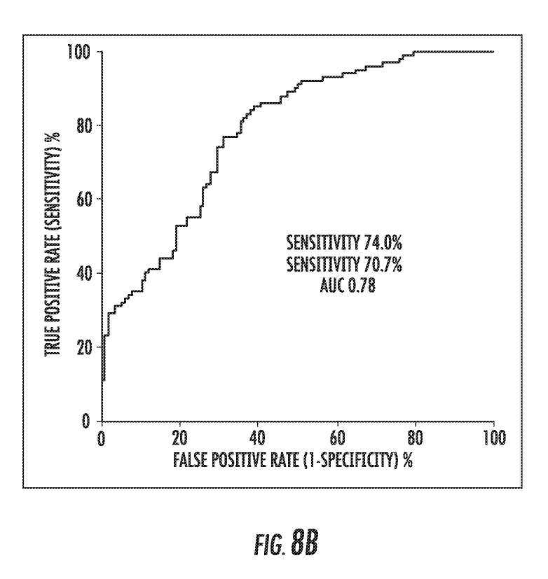

FIG. 8A is plot of a value of single ions versus retention time on the chromatogram. FIG. 8B displays a subset of the 544 mass ion biomarkers of lung cancer (i.e. those with the highest C-statistic values that were identified by Monte Carlo statistical analysis in the unblinded-phase. M/z is the mass divided by the charge number of an ion, and the retention time indicates when a VOC eluted from the GC column and entered the MS detector where it was bombarded with electrons and converted to mass ion fragments. Vertical linear groups of single ions with similar retention times between 2,000 and 2,500 sec are shown. These groupings are consistent with one or more breath VOCs entering the MS detector in a single peak prior to breakdown to mass ions. It was found that a comparatively small number of parent breath VOCs may account for the majority of the mass ion biomarkers of lung cancer.

It was found that in the unblinded model-building phase, the method of the present invention identified lung cancer with sensitivity 74.0%, specificity 70.7% and C-statistic 0.78 as shown in FIG. 8B. In the blinded model-testing phase, the method predicted lung cancer at Laboratory A with sensitivity 68.0%, specificity 68.4%, C-statistic 0.71; and at Laboratory B with sensitivity 70.1%, specificity 68.0%, C-statistic 0.70, with linear correlation between replicates (r=0.88). It is projected that the combination of the method of the present invention for breath testing to detect lung cancer in parallel with chest CT can improve the sensitivity and specificity of chest CT, reducing false-positives by 66.2% and false-negatives by 71.0%.

FIG. 9A is a graph of DF values at laboratory A versus DF values at laboratory B. Chromatograms analyzed at laboratory A were plotted as a function of the DF value of the duplicate sample analyzed at laboratory B line 400 shows a linear relationship between the two sets of DF values (r=0.88).

FIG. 9B is a graph of predicted sensitivity and specificity in subjects with biopsy-proven lung cancer and chest CT negative for lung cancer in the blinded-phase at Laboratory A. The DF value derived from the predictive algorithm provides a variable cutoff point for the breath test. Test results greater than a DF value were scored as positive for lung cancer while those less than the DF were scored as negative. When DF=0, sensitivity curve 401 shows 100% sensitivity because all results are scored as positive for lung cancer and specificity curve 402 shows zero specificity because no results are scored as negative during performing block 206. Point 404 where sensitivity curve 401 and specificity curve 402 intersect generally yields the optimal DF value for a binary test to detect lung cancer, as cancer versus no cancer. Sensitivity curve 401 and specificity curve 402 intersected at DF=22, with sensitivity 68.0% and specificity 68.4%.

FIG. 9C is a graph of the value of true positives and true negatives versus the discriminant function (DF). FIG. 9C demonstrates that the risk of lung cancer varied with the value of the discriminant function (DF). As the DF value increased, the cumulative percentage of true positive results (1-sensitivity) shown in curve 410 rose while the cumulative percentage of true negatives (1-specificity) shown in curve 412 fell. Assignment of lung cancer risk can be determined as a function of DF, such that for example when DF>40, more than 50% of subjects had lung cancer, while at DF<18, more than 50% of subjects were cancer free.

FIG. 9D a graph of receiver operating characteristic (ROC) curves for predicting lung cancer in the blinded-phase B for performing block 206. ROC curve 500 is shown for samples analyzed at laboratory A. ROC curve 502 is shown for samples analyzed at laboratory B. The overall accuracy (C-statistic) of the lung cancer predictions was similar at both sites (71% and 70%).

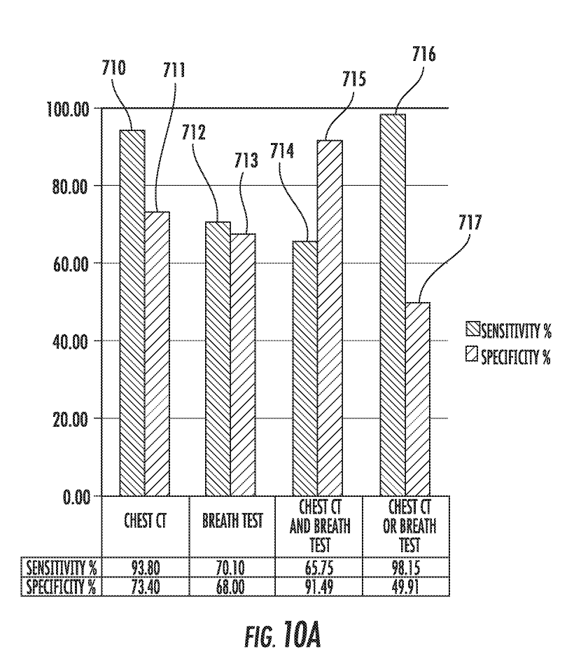

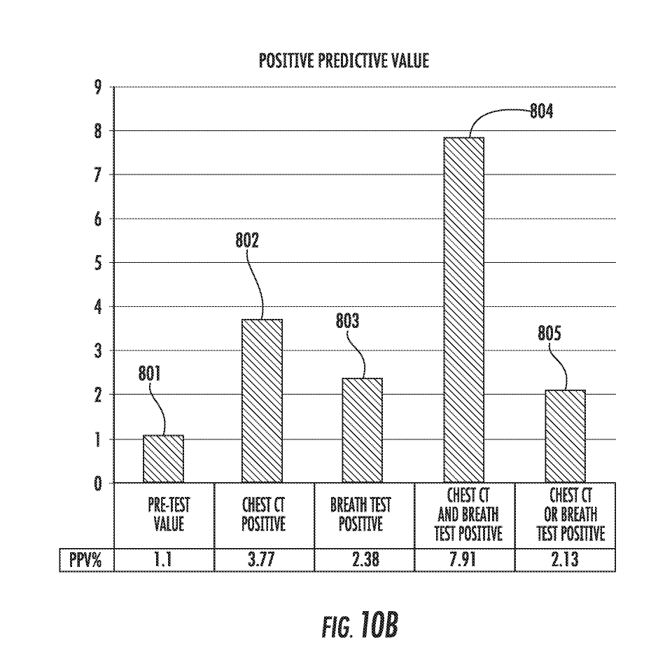

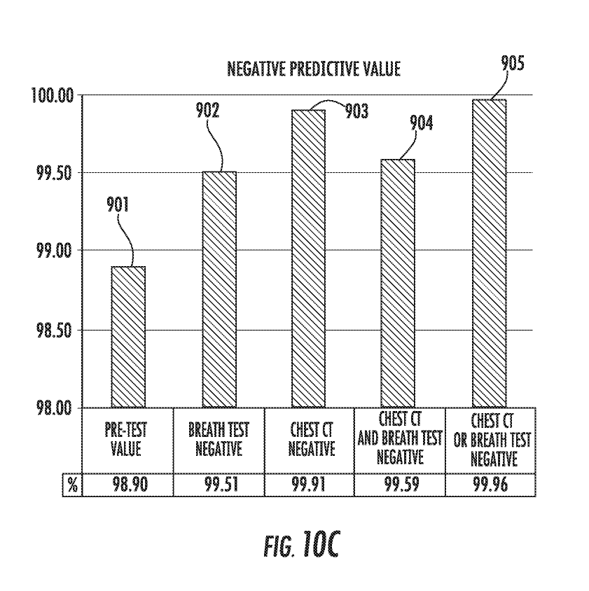

FIG. 10A is a graph of expected outcome of chest CT combined with breath testing. Block 710 represents sensitivity % for chest CT. Block 711 represents specificity % for chest CT. These predictions employ values reported in the National Lung Screening Trial for lung cancer prevalence (1.1%) and screening chest CT (sensitivity 93.8%, specificity 73.4%). Block 712 represents sensitivity % for a breath test performed by method 10 of the present invention. Block 713 represents specificity % for a breath test performed by method 10 of the present invention. Block 714 represents sensitivity % for the combination of a chest CT and a breath test performed by method 10 of the present invention. Block 715 represents specificity % for the combination of a chest CT and breath test performed by method 10 of the present invention. Block 716 represents sensitivity % for the combination of a chest CT or a breath test performed by method 10 of the present invention. Block 717 represents specificity % for the combination of a chest CT or a breath test performed by method 10 of the present invention.

This figure displays the expected improvement in sensitivity and specificity of chest CT for lung cancer if it is combined in parallel with a breath testing. If both tests are positive for lung cancer, then specificity increases from 73.4% to 91.49%. If either test is positive, then sensitivity increases from 93.8% to 98.15%. These improvements were computed from the formulas for combining two independent tests (A and B) in parallel: If both tests are positive, then sensitivity (sen)=(A)ₛₑₙ×(B)ₛₑₙ, and specificity (spec)=(A)_{spec}+(B)_{spec}−[(A)_{spec}×(B)_{spec}]. Compared to either test employed alone, their combined specificity is increased but sensitivity is reduced. If only one of the tests is positive, then sensitivity=(A)ₛₑₙ+(B)ₛₑₙ [−(A)ₛₑₙ×(B)ₛₑₙ] and specificity=(A)_{spec}×(B)_{spec}. Compared to either test employed alone, their combined sensitivity is increased but specificity is reduced. FIG. 10A demonstrates that the sensitivity and specificity of the two tests employed in combination are greater than the sensitivity and specificity of either test when employed alone.

FIG. 10B is a graph of a positive predictive value (PPV) of chest CT combined with breath testing. This figure displays the expected improvement in PPV of chest CT for lung cancer if combined in parallel with a breath test. Block 801 shows a pre-test value. Employed alone, the PPV of chest CT is 3.77% as shown in block 802 and the PPV of the breath test is 2.38% as shown in block 803. If breath testing performed according to method 10 is employed in parallel with chest CT and both tests are positive, then the PPV increases to 7.91% as shown in block 804, i.e. it increases by a factor of 2.1. The improvement is due to the higher specificity of the combined test and the consequent reduction in false positive results. The PPV of a test depends upon the prevalence (prev) of a disease, and is computed as PPV=(sen×prev)/[(sen×prev+(1−spec)×(1−prev)]. The PPV of chest CT for lung cancer is 3.77% [i.e. 0.938×011/(0.938×011+(1−0.734×(1−0.011))=0.0377]. If breath testing or chest CT is positive, the PPV is 2.13% as shown in block 805.

FIG. 10C is a graph of a negative predictive value (NPV) of chest CT combined with breath testing. Block 901 shows a pre-test value. The NPV for breath testing is shown in block 902 and the NPV for a chest CT is shown in block 903. The NPV of the chest CT and the breath testing is shown in block 904. The NPV of the chest CT or the breath testing is shown in block 905. When either of the tests are negative, the NPV would be increased from 99.52% with chest CT alone to 99.96%. Despite the increased sensitivity of the combined test, only a modest increment in NPV is possible because the pre-test NPV based on prevalence of lung cancer is 98.9%.

Expected outcome of screening one million people for lung cancer is shown in table 3.

TABLE 3

| | sensi- | speci- | | | | |
|---|---|---|---|---|---|---|
| | tivity | ficity | TP | FN | TN | FP |
| Chest CT | 93.80 | 73.40 | 10,318 | 682 | 725,926 | 263,074 |
| Breath test | 71.00 | 66.20 | 7,810 | 3,190 | 654,718 | 334,282 |
| Chest CT AND | 66.60 | 91.01 | 7,326 | 3,674 | 900,081 | 88,919 |
| breath test | | | | | | |
| Chest CT OR | 98.20 | 48.59 | 10,802 | 198 | 480,583 | 508,437 |
| breath test | | | | | | |

Table 3 indicates TP=true positives, FN=false negatives, TN=true negatives, and FP=false positives. The main limiting factor in population screening programs is the potentially overwhelming number of false-positive test results. Screening one million people with chest CT alone would result in 263,074 false positive test results, but if chest CT and breath testing are positive, the increased specificity would reduce this number to 88,919 i.e. by 66.2%. However, if only one of the tests is positive, then the increased sensitivity would reduce the number of false-negatives from 682 to 198 i.e. by 71.0%.

The present results indicate that ionic biomarkers in breath accurately predicted the presence or absence of lung cancer in a blinded validation study. A multivariate algorithm predicted the diagnosis from replicate breath samples independently analyzed at two laboratories, and the sensitivity, specificity, and overall accuracy of the test were similar at both sites. The outcome of the test was not significantly affected by age or pack-years of tobacco smoking.

The breath test for biomarker ions can improve both the sensitivity and the specificity of chest CT if the two tests are employed in parallel. In a program to screen one million asymptomatic high risk-subjects for lung cancer with chest CT alone, the expected outcome would include 263,074 false-positive test results. However, if chest CT and a breath test are combined in parallel, the number of false-positive results would be expected to fall to 88,919, a reduction of 66.2%. Similarly, if only one of the tests is positive, then the number of false-negatives would be expected to fall from 682 to 198 i.e. by 71.0%. As a result, combined parallel testing could potentially facilitate large-scale screening for lung cancer by reducing the economic costs and the potential harms of false-positive and false-negative test outcomes that are currently associated with chest CT.

Model-Building Phase—Unblinded for Detecting Breast Cancer

Collection of breath VOC samples: Collection of breath VOC samples was performed in accordance with method 10 for identifying biomarkers and generating an output indicative of lung cancer and system 60. A subject wears a nose clip and breathes normally through a disposable valved mouthpiece and bacterial filter into the BCA for 2.0 min. Alveolar breath VOCs are captured on to a sorbent trap that is immediately sealed in a hermetic container.

VOCs in 54 women with biopsy-proven breast cancer and in 204 healthy controls were analyzed. Subjects were randomly assigned to a training set (2/3) and a test set (1/3). Analysis of breath VOC samples: Analysis of breath VOC sample was performed with method 10 for identifying biomarkers and generating an output indicative of lung cancer and system 60. Using automated instrumentation, VOCs were thermally desorbed from the sorbent trap 62, cryogenically concentrated, and assayed by gas chromatography mass spectrometry (GC MS). A known quantity of an internal standard (bromofluorobenzene) was automatically loaded on to all samples in order to normalize the abundance of VOCs and to facilitate alignment of chromatograms.

Analysis of data: GC MS data from both laboratories was pooled for analysis and development of a single predictive algorithm.

Alignment of single ion masses in chromatograms: Chromatograms were processed with metabolomic analysis software (XCMS in R) in order to generate a table listing retention times with their associated ion masses and intensities, and binned into a series of 5 sec retention time segments. In the training set, mass ions in each time segment were ranked according to their diagnostic accuracy i.e. the area under curve (AUC) of the receiver operating characteristic (ROC) curve. Correct assignment curve 50b shown in FIG. 4B displays the number of mass ions as a function of their diagnostic accuracy, defined as the area under curve (AUC) of its associated receiver operating characteristic (ROC) curve. Random assignment curve 52b similarly displays the number of mass ions as a function of their diagnostic accuracy employing the mean of 50 random assignments of diagnosis (“cancer” or “cancer-free”). Where the random assignment curve fell to zero at approximately AUC=0.6, 21 mass ions in correct assignment curve 50b exhibited diagnostic accuracy that was superior to random behavior.

Multiple Monte Carlo simulations were employed in order to minimize the risk of including random identifiers of disease by selecting the mass ions in each time segment that identified active breast cancer with greater than random accuracy and combined those with the highest diagnostic accuracy in a predictive algorithm using multivariate weighted digital analysis (WDA). The algorithm was used to predict the diagnosis in the test set.

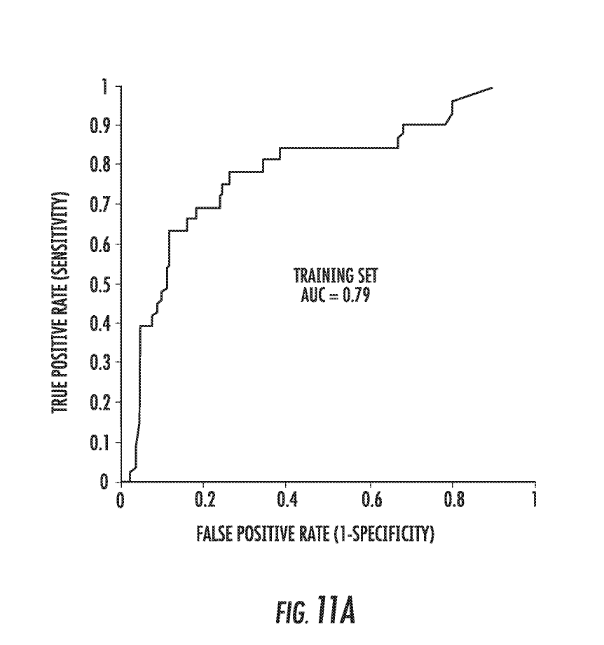

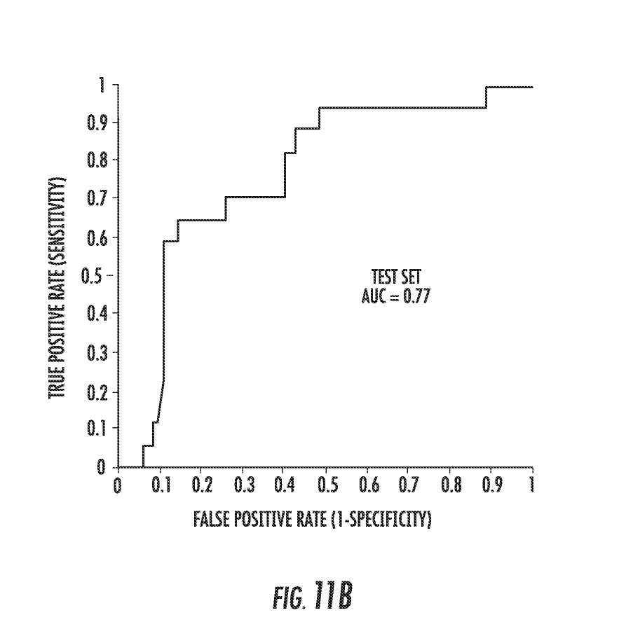

It was found that the method of the present invention using the WDA algorithm employing 21 mass ion biomarkers identified breast cancer with sensitivity 79.0% in the training set as shown in FIG. 11A and with sensitivity 79.0% in the test set as shown in FIG. 11B. Breath mass ions biomarkers accurately identified women with breast cancer and could potentially be used in early diagnosis and treatment monitoring.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

### REFERENCES

The following references, to the extent that they provide exemplary procedural or other details supplementary to those set forth herein, are specifically incorporated herein by reference.
1. Pauling L, Robinson A B, Teranishi R, Cary P. Quantitative analysis of urine vapor and breath by gas-liquid partition chromatography. Proc Natl Acad Sci USA 1971; 68:2374-6.
2. Silkoff P E, Carlson M, Bourke T, Katial R, Ogren E, Szefler S J. The Aerocrine exhaled nitric oxide monitoring system NIOX is cleared by the US Food and Drug Administration for monitoring therapy in asthma. J Allergy Clin Immunol 2004; 114:1241-56.
3. Gordon S M, Szidon J P, Krotoszynski B K, Gibbons R D, O'Neill H J. Volatile organic compounds in exhaled air from patients with lung cancer. Clin Chem 1985; 31:1278-82.
4. Phillips M, Gleeson K, Hughes J M, et al. Volatile organic compounds in breath as markers of lung cancer: a cross-sectional study. Lancet 1999; 353:1930-3.
5. Phillips M, Altorki N, Austin J H, et al. Prediction of lung cancer using volatile biomarkers in breath. Cancer Biomark 2007; 3:95-109.
6. Preti G L J, Kostelc J G, Aldinger S, Daniele R. Analysis of lung air from patients with bronchogenic carcinoma and controls using gas chromatography-mass spectrometry. J Chromatogr 1988; 432:1-11.
7. Bousamra M, 2nd, Schumer E, Li M, et al. Quantitative analysis of exhaled carbonyl compounds distinguishes benign from malignant pulmonary disease. J Thorac Cardiovasc Surg 2014; 148:1074-80; discussion 80-1.
8. Adiguzel Y, Kulah H. Breath sensors for lung cancer diagnosis. Biosens Bioelectron 2014; 65C:121-38.
9. Peng G, Hakim M, Broza Y Y, et al. Detection of lung, breast, colorectal, and prostate cancers from exhaled breath using a single array of nanosensors. Br J Cancer 2010; 103:542-51.
10. Mozzoni P, Banda I, Goldoni M, et al. Plasma and EBC microRNAs as early biomarkers of non-small-cell lung cancer. Biomarkers 2013; 18:679-86.
11. Carpagnano G E, Lacedonia D, Spanevello A, et al. Exhaled breath temperature in NSCLC: could be a new non-invasive marker? Med Oncol 2014; 31:952.
12. Boedeker E, Friedel G, Walles T. Sniffer dogs as part of a bimodal bionic research approach to develop a lung cancer screening. Interact Cardiovasc Thorac Surg 2012; 14:511-5.
13. Phillips M, Cataneo R N, Chaturvedi A, et al. Detection of an Extended Human Volatome with Comprehensive Two-Dimensional Gas Chromatography Time-of-Flight Mass Spectrometry. PLoS One 2013; 8:e75274.
14. Phillips M, Byrnes R, Cataneo R N, et al. Detection of volatile biomarkers of therapeutic radiation in breath. J Breath Res 2013; 7:036002.
15. Miekisch W, Herbig J, Schubert J K. Data interpretation in breath biomarker research: pitfalls and directions. J Breath Res 2012; 6:036007.
16. van der Schee M P, Paff T, Brinkman P, van Aalderen W M, Haarman E G, Sterk P J. Breathomics in lung disease. Chest 2015; 147:224-31.
17. Centers for Disease Control & Prevention: Lung Cancer Statistics. http://wwwcdcgov/cancer/lung/statistics/.
18. National Lung Screening Trial Research T, Church T R, Black W C, et al. Results of initial low-dose computed tomographic screening for lung cancer. N Engl J Med 2013; 368:1980-91.
19. Aberle D R, DeMello S, Berg C D, et al. Results of the two incidence screenings in the National Lung Screening Trial. N Engl J Med 2013; 369:920-31.
20. Jeffers C D, Pandey T, Jambhekar K, Meek M. Effective use of low-dose computed tomography lung cancer screening. Curr Probl Diagn Radiol 2013; 42:220-30.
21. Carlile P V. Lung cancer screening: where have we been? Where are we going? The Journal of the Oklahoma State Medical Association 2015; 108:14-8.
22. Sather M R, Raisch D W, Haakenson C M, Buckelew J M, Feussner J R, Department of Veterans Affairs Cooperative Studies P. Promoting good clinical practices in the conduct of clinical trials: experiences in the Department of Veterans Affairs Cooperative Studies Program. Control Clin Trials 2003; 24:570-84.
23. Phillips M. Method for the collection and assay of volatile organic compounds in breath. Anal Biochem 1997; 247:272-8.
24. Mente S, Kuhn M. The use of the R language for medicinal chemistry applications. Curr Top Med Chem 2012; 12:1957-64.
25. Gowda H, Ivanisevic J, Johnson C H, et al. Interactive XCMS Online: simplifying advanced metabolomic data processing and subsequent statistical analyses. Anal Chem 2014; 86:6931-9.
26. Phillips M, Basa-Dalay V, Bothamley G, et al. Breath biomarkers of active pulmonary tuberculosis. Tuberculosis (Edinb) 2010; 90:145-51.
27. Phillips M, Altorki N, Austin J H, et al. Detection of lung cancer using weighted digital analysis of breath biomarkers. Clin Chim Acta 2008; 393:76-84.
28. Weinstein S, Obuchowski N A, Lieber M L. Clinical evaluation of diagnostic tests. AJR Am J Roentgenol 2005; 184:14-9.
29. Stein S. Mass spectral reference libraries: an ever-expanding resource for chemical identification. Anal Chem 2012; 84:7274-82.
30. Handa H, Usuba A, Maddula S, Baumbach J I, Mineshita M, Miyazawa T. Exhaled breath analysis for lung cancer detection using ion mobility spectrometry. PLoS One 2014; 9:e114555.
31. Westhoff M, Litterst P, Freitag L, Urfer W, Bader S, Baumbach J I. Ion mobility spectrometry for the detection of volatile organic compounds in exhaled breath of patients with lung cancer: results of a pilot study. Thorax 2009; 64:744-8.
32. Hakim M, Broza Y Y, Barash 0, et al. Volatile Organic Compounds of Lung Cancer and Possible Biochemical Pathways. Chem Rev 2012.
33. Filipiak W, Filipiak A, Sponring A, et al. Comparative analyses of volatile organic compounds (VOCs) from patients, tumors and transformed cell lines for the validation of lung cancer-derived breath markers. J Breath Res 2014; 8:027111.

What is claimed is:

1. A method for identifying a plurality of biomarkers for predicting disease in a subject which comprises the steps of:
a. collecting a breath sample from subjects known to have a disease and subjects known to be free of the disease;
b. analyzing the collected breath samples to determine all mass ions in each of the collected breath samples using at least one time resolved separation technique and at least one mass resolved separation technique;
c. identifying a subset of the determined mass ions in a processor as the biomarkers for detecting disease, the subset of the determined mass ions are statistically significant for detecting the disease; and
d. combining the subset of the determined mass ions in a multivariate algorithm in a processor to generate a discriminant function,
wherein the discriminant function indicates a value of the likelihood that the subject has the disease.

2. The method of claim 1 wherein the subjects are human.

3. The method of claim 1 wherein the disease is breast cancer.

4. The method of claim 1 wherein the at least one time resolved separation technique is gas chromatography.

5. The method of claim 1 wherein the at least one mass resolved separation technique is mass spectrometry.

6. The method of claim 1 wherein in step c. of identifying a subset of the determined mass ions further includes the steps of:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting the disease; and
selecting the candidate biomarker mass ions with at least greater than random diagnostic accuracy as the subset of the determined mass ions which are statistically significant for detecting the disease.

7. The method of claim 6 wherein the step of ranking candidate biomarker mass ions by diagnostic accuracy is determined by the steps of:
determining a receiver operating characteristic (ROC) curve for each of the candidate biomarker mass ions;
evaluating an area under the ROC curve for each of the candidate biomarker mass ions reflecting the diagnostic accuracy for detecting disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.

8. The method of claim 7 wherein the correct assignment curve and the random assignment curve are generated using Monte Carlo analysis.

9. The method of claim 7 wherein the subset of candidate biomarker mass ions with greater than random ability to identify the disease is identified from a vertical line V₁ at the point where the value of the random assignment curve is zero.

10. The method of claim 9 wherein the area under the ROC curve for the subset of candidate biomarker mass ions is at least 0.6.

11. The method of claim 7 further comprising:
a display and further comprising:
controlling the display to display the subset of candidate biomarker mass ions by the processor.

12. A method for detecting the probable presence of a disease in a test subject which comprises the steps of:
a. collecting a breath sample from subjects known to have the disease and subjects known to be free of the disease;
b. analyzing the collected breath samples to determine all mass ions in each of the collected breath samples using at least one time resolved separation technique and at least one mass resolved separation technique;
c. identifying a subset of the determined mass ions in a processor as the biomarkers for detecting disease, the subset of the determined mass ions are statistically significant for detecting the disease;
d. combining the subset of the determined mass ions in a multivariate algorithm in a processor to generate a first value of a discriminant function;
e. collecting a breath sample of the test subject;
f. analyzing the collected breath sample of the test subject to determine all mass ions in breath of the test subject using at least one time resolved separation technique and at least one mass resolved separation technique;
g. combining the mass ions determined for the test subject in the multivariate algorithm to generate a second value of the discriminant function; and
h. comparing the first value of the discriminant function to the second value of the discriminant function, wherein when the second value of the discriminant function is the same or larger than the first value of the discriminant function indicating a first probability of the presence of the disease in the test subject.

13. The method of claim 12 wherein the subjects are human.

14. The method of claim 12 wherein the disease is breast cancer.

15. The method of claim 12 wherein the at least one time resolved separation technique is gas chromatography.

16. The method of claim 12 wherein the at least one mass resolved separation technique is mass spectrometry.

17. The method of claim 12 wherein in step c. of identifying a subset of the determined mass ions further includes the steps of:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting the disease; and
selecting the candidate biomarker mass ions with at least greater than random diagnostic accuracy as the subset of the determined mass ions which are statistically significant for detecting the disease.

18. The method of claim 17 wherein the step of ranking candidate biomarker mass ions by diagnostic accuracy is determined by the steps of:
determining a receiver operating characteristic (ROC) curve for each of the candidate biomarker mass ions;
evaluating an area under the ROC curve for each of the candidate biomarker mass ions reflecting the diagnostic accuracy for detecting the disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.

19. The method of claim 18 wherein the correct assignment curve and the random assignment curve are generated using Monte Carlo analysis.

20. The method of claim 19 wherein the subset of candidate biomarker mass ions with greater than random ability to identify disease is identified from a vertical line V₁ at the point where the value of the random assignment curve is zero.

21. The method of claim 20 wherein the area under the ROC for each of the selected candidate biomarker mass ions is at least 0.6.

22. The method of claim 12 further comprising the step of screening the subject with a chest computed tomography (CT) scan for determining a second probability for detecting disease in the subject; and combining the first probability with the second probability to determine a resultant probability of predicting disease.

23. A system for identifying a plurality of biomarkers for predicting disease in a subject which comprises:
an apparatus for collecting a breath sample from subjects known to have a disease and subjects known to be free of the disease;
mass spectrometer (MS) associated with a gas chromatograph (GC) apparatus for analyzing the collected breath samples to determine all mass ions in each of the collected breath samples;
a computer that identifies a subset of the determined mass ions as the biomarkers for detecting the disease, the subset of the determined mass ions are statistically significant for detecting the disease and combines the subset of the determined mass ions in a multivariate algorithm to generate a discriminate function, wherein the discriminate function indicates a value of the likelihood that the subject has the disease.

24. The system of claim 23 wherein the subset of the determined mass ions is identified by:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting disease; and
selecting the candidate biomarker mass ions with at least greater than random diagnostic accuracy as the subset of the determined mass ions which are statistically significant for detecting the disease.

25. The system of claim 24 wherein candidate biomarker mass ions are ranked by diagnostic accuracy is determined by:
determining a receiver operating characteristic (ROC) curve for each of the candidate biomarker mass ions;
evaluating an area under the ROC curve for each of the candidate biomarker mass ions reflecting the diagnostic accuracy for detecting the disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.

26. The system of claim 25 wherein the correct assignment curve and the random assignment curve are generated using Monte Carlo analysis.

27. The system of claim 25 wherein the subset of candidate biomarker mass ions with greater than random ability to identify disease is identified from a vertical line V₁ at the point where the value of the random assignment curve is zero.

28. The system of claim 25 wherein the disease is breast cancer.

29. A system for predicting disease in a test subject which comprises:
an apparatus for collecting a breath sample from the test subject;
mass spectrometer (MS) associated with a gas chromatograph (GC) apparatus for analyzing the collected breath sample from the test subject to determine all mass ions;
a computer that identifies a subset of determined mass ions as the biomarkers for detecting disease from a data set of mass ions of subjects known to have the disease and subjects known to be free of the disease, the subset of the determined mass ions are statistically significant for detecting the disease, combines the subset of the determined mass ions in a multivariate algorithm to generate a first value of a discriminate function, combines the mass ions determined for the test subject in the multivariate algorithm to generate a second value of the discriminate function and compares the first value to the second value, wherein when the second value is the same or larger than the first value indicating the probable presence of the disease.

30. The system of claim 29 wherein the subset of the determined mass ions is identified by:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting disease; and
selecting the candidate biomarker mass ions with at least greater than random diagnostic accuracy as the subset of the determined mass ions which are statistically significant for detecting disease.

31. The system of claim 29 wherein the candidate biomarker mass ions are ranked by diagnostic accuracy by:
determining a receiver operating characteristic (ROC) curve for each of the candidate biomarker mass ions;
evaluating an area under the ROC curve for each of the candidate biomarker mass ions reflecting the diagnostic accuracy for detecting the disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.

32. The system of claim 29 wherein the correct assignment curve and the random assignment curve are generated using Monte Carlo analysis.

33. The system of claim 29 wherein the subset of candidate biomarker mass ions with greater than random ability to identify disease is identified from a vertical line V₁ at the point where the value of the random assignment curve is zero.

34. The system of claim 29 further comprising a display and controlling the display to display the subset of candidate biomarker mass ions by the processor.

35. A computer program product comprising at least one non-transitory computer readable medium storing instructions translatable by a computer to perform:
analyzing collected breath samples from subjects known to have a disease and subjects known to be free of the disease to determine all mass ions in each of the collected breath samples using at least one time resolved separation technique and at least one mass resolved separation technique;
identifying a subset of the determined mass ions as the biomarkers for detecting disease, the subset of the determined mass ions are statistically significant for detecting the disease;
combining the subset of the determined mass ions in a multivariate algorithm in a processor to generate a discriminant function; and
returning a value of the discriminant function to indicate the likelihood that the subject has the disease.

36. The computer program product of claim 35 wherein the instructions are further translatable to perform identifying the subset of the determined mass ions by:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting the disease; and
selecting the candidate biomarker mass ions with at least greater than random diagnostic accuracy as the subset of the determined mass ions which are statistically significant for detecting the disease.

37. The computer program product of claim 35 wherein the instructions are further translatable to perform ranking candidate biomarker mass ions by diagnostic accuracy is determined by:
determining a receiver operating characteristic (ROC) curve for each of the candidate biomarker mass ions;
evaluating an area under the ROC curve for each of the candidate biomarker mass ions reflecting the diagnostic accuracy for detecting the disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.

38. The computer program product of claim 35 wherein the instructions are further translatable to perform combining a probability of detecting disease with a chest computed tomography (CT) scan with the probability of the likelihood that subject has disease determined by the value of the discriminant function.
